(19) United States
(12) Patent Application Publication
(10) Pub. No.: US 2007/0031920 A1
(43) Pub. Date: Feb. 8, 2007

(54) High expression locus vector based on ferritin heavy chain gene locus

(75) Inventor: Holly Prentice, Carlisle, MA (US)

Correspondence Address: STERNE, KESSLER, GOLDSTEIN & FOX, P.L.L.C., 1100 NEW YORK AVE., N.W., WASHINGTON, DC, 20005, US

(73) Assignee: BIOGEN, INC., Cambridge, MA (US)

(21) Appl. No.: 10/532,197

(22) PCT Filed: Oct. 22, 2003
(86) PCT No.: PCT/US03/33433

§ 371 Date: Sep. 12, 2006

Related U.S. Application Data
(60) Provisional application No. 60/421,252, filed on Oct. 24, 2002.

(51) Int. Cl. C12P 21/06; C07H 21/04; C07K 14/80; C12N 15/09

(52) U.S. Cl. 435/69.1; 435/455; 435/325; 530/400; 536/23.5

(57) ABSTRACT

High expression locus vectors based, in part, on the ferritin heavy chain locus are disclosed. The vectors include distal 5′ flanking sequences and/or proximal 5′ regulatory sequences derived from ferritin heavy chain locus. The vectors include a site for insertion of heterologous sequences and proximal 3′ regulatory and distal 3+ flanking sequences. The proximal 3′ regulatory and distal 3′ flanking sequences are optionally derived from the ferritin heavy chain locus. Cells transformed with the vectors, and methods of producing heterologous proteins encoded by the vectors, are also disclosed.

### BACKGROUND OF THE INVENTION

1. Field of the Invention

This invention relates to the field of molecular biology, and in particular to the development and use of vectors for the expression of heterologous genetic sequences in transformed cells.

2. Description of the Related Art

Typical expression vectors contain promoters to drive the gene of interest as well as polyadenylation signals to generate a mature transcript. Promoter sequences tend to be only a few hundred base pairs in length and contain most, if not all, of the regulatory regions for optimal expression as determined by transient transfection. However, expression constructs containing these sequences, although highly functional in transient transfections, are not always able to confer a similar level of expression when integrated into the chromatin as a stable transfectant. This is due to position-dependent expression, a phenomenon in which the site of integration has a dominant effect, usually negative, on the level of expression (Wilson (1990), Ann. Rev. Cell Biol. 6:679-714). The result of position-dependent expression is evident in the results of a transfection screening, in which most of the cell lines produce little or no product. Therefore, it is usually necessary to screen a large number of transfectants in order to identify a single high-expressing clone. Even after extensive screening, transfectants obtained using standard expression vectors typically have expression levels that would not be sufficient to meet commercial titer goals.

The time consuming and labor intensive process of DHFR amplification is frequently employed to increase expression levels in stable transfectants. For example, integrated copies of standard expression constructs typically require amplification to greater than 100 copies in order to approach the level of expression of endogenous genes with promoters of similar strength (from only two alleles). The differences between standard expression vectors and endogenous genes are most likely due to the presence of sequences 5′ to the promoter and/or 3′ to the are most likely due to the presence of sequences 5′ to the promoter and/or 3′ to the polyadenylation signal of the endogenous genes that are able to confer a chromatin configuration more favourable for expression. An expression construct containing sequences that can confer favourable position-independent chromatin configurations, regardless of the integration site(s) would be advantageous for generating cell lines highly expressing heterologous genes.

### SUMMARY OF THE INVENTION

The present invention depends, in part, upon the development of high expression “locus vectors” derived from the ferritin heavy chain gene. The concept of a “locus vector” is based on the observation that the regions found 5′ and 3′ to highly expressed genes in their natural chromatin contexts can confer higher levels of expression to a heterologous gene. Therefore, the present invention provides ferritin heavy chain gene locus vectors which include 5′ and 3′ sequences which can convey high levels of expression to heterologous genes in stable transfectants. Thus, the invention provides genetic vectors for the stable transfection and expression at high levels of a desired protein within eukaryotic cells.

In one aspect, the invention provides genetic vectors for stable transfection and expression of a desired protein within eukaryotic cells including: (a) distal 5′ flanking sequences of a eukaryotic locus; (b) proximal 5′ regulatory sequences of a eukaryotic locus; (c) at least a first insertion site for a heterologous sequence; and (d) proximal 3′ regulatory sequences effective for transcription termination of a eukaryotic locus; in which these sequences are operablyjoined in the order (a)-(d) in a 5′ to 3′ orientation, with optional linker sequences between adjacent sequences; and in which (1) the distal 5′ flanking sequences comprise a sequence of at least 100 bases having at least 70% identity to a nucleotide sequence found between 20 bp and 100,000 bp 5′ of a transcriptional initiation site of a ferritin heavy chain locus; and/or (2) the proximal 5′ regulatory sequences comprise a sequence of at least 20 bases having at least 70% identity to a nucleotide sequence found between 1 bp and 10,000 bp 5′ of a translational initiation codon of a ferritin heavy chain locus.

In another aspect, the vector includes at least a first heterologous coding sequence encoding a desired protein. Thus, the invention provides genetic vectors for stable transfection and expression of a desired protein within eukaryotic cells including: (a) distal 5′ flanking sequences of a eukaryotic locus; (b) proximal 5′ regulatory sequences of a eukaryotic locus; (c) at least a first heterologous coding sequence encoding said desired protein; and (d) proximal 3′ regulatory sequences effective for transcription termination of a eukaryotic locus; in which these sequences are operably joined in the order (a)-(d) in a 5′ to 3′ orientation, with optional linker sequences between adjacent sequences; and in which (1) the distal 5′ flanking sequences comprise a sequence of at least 1 00 bases having at least 70% identity to a nucleotide sequence found between 20 bp and 100,000 bp 5′ of a transcriptional initiation site of a ferritin heavy chain locus; and/or (2) the proximal 5′ regulatory sequences comprise a sequence of at least 20 bases having at least 70% identity to a nucleotide sequence found between 1 bp and 10,000 bp 5′ of a translational initiation codon of a ferritin heavy chain locus.

In some embodiments, the distal 5′ flanking sequences are derived from a ferritin heavy chain locus. In other embodiments, the proximal 5′ regulatory sequences are derived from a ferritin heavy chain locus. In yet other embodiments, both the proximal 5′ regulatory sequences and the distal 5′ flanking sequences are derived from a ferritin heavy chain locus.

In some embodiments, the proximal 3′ regulatory sequences are derived from a ferritin heavy chain locus, and in some embodiments the vector further includes distal 3′ flanking sequences of a ferritin heavy chain locus.

In certain embodiments of the invention, the insertion site for a heterologous sequence includes at least one restriction endonuclease site, and in other embodiments the insertion site for a heterologous sequence is a polylinker site including at least two restriction endonuclease sites.

In certain embodiments of the invention, the proximal 5′ regulatory sequences include a eukaryotic intron sequence. In some of these embodiments, the eukaryotic intron sequence is derived from intron 1 of a ferritin heavy chain gene. In certain embodiments, the proximal 5′ regulatory sequences include untranslated exon sequences.

In some embodiments, the distal 5′ flanking sequences and the proximal 5′ regulatory sequences have a total length of between 1,000 and 10,000 bases. Similarly, in some embodiments, the proximal 3′ regulatory sequences and any distal 3′ flanking sequences have a total length of between 1,000 and 10,000 bases.

In another aspect, the invention provides eukaryotic cells transfected with any of the vectors of the invention. In some embodiments, the vector has stably integrated into a chromosome of said cell and, in some embodiments, the first heterologous coding sequence is expressed in said cell.

In some embodiments, the invention provides eukaryotic cells including: (a) distal 5′ flanking sequences of a eukaryotic locus; (b) proximal 5′ regulatory sequences of a eukaryotic locus; (c) at least a first coding sequence; and (d) proximal 3′ regulatory sequences effective for transcription termination of a eukaryotic locus; in which the sequences are operably joined in order (a)-(d) in a 5′ to 3′ orientation, with optional linker sequences between adjacent sequences; and in which (1) the distal 5′ flanking sequences comprise an exogenous sequence of at least 100 bases having at least 70% identity to a nucleotide sequence found between 20 bp and 100,000 bp 5′ of a transcriptional initiation site of a ferritin heavy chain locus; and/or (2) the proximal 5′ regulatory sequences comprise an exogenous sequence of at least 20 bases having at least 70% identity to a nucleotide sequence found between 1 bp and 10,000 bp 5′ of a translational initiation codon of a ferritin heavy chain locus.

In another aspect, the invention provides a eukaryotic cell including an exogenous 5′ distal flanking sequence derived from a ferritin heavy chain locus operably joined to a coding sequence.

In another aspect, the invention provides a method of producing a desired protein in a eukaryotic cell including the steps of (a) providing at least one cell of the invention or a descendent thereof; (b) maintaining the cell in a culture under conditions which permit high expression of the desired protein; and (c) isolating the desired protein from the culture.

These and other aspects and advantages of the invention will be apparent to those of skill in the art from the detailed description and examples which follow.

### BRIEF DESCRIPTION OF THE DRAWINGS

The following drawings are illustrative of embodiments of the invention and are not meant to limit the scope of the invention as encompassed by the claims.

FIG. 1 shows rat ferritin heavy chain exon sequences.

FIG. 2 illustrates one example of the subcloning of the region containing the ferritin heavy chain exons into the Litmus 38 plasmid.

FIG. 3 illustrates the deletion of exons 2, 3, and 4 from pFerX1 and insertion of a polylinker to generate plasmid pFerX2.

FIG. 4 illustrates the deletion of the exon 1 coding region from pFerX2 to generate plasmid pFerX3, and deletion of the IRE to generate plasmid pFerX4.

FIG. 5 A-B illustrates the removal of exons 2 through 4 of the ferritin heavy chain gene from cosmid 15A using PCR fusion.

FIG. 6 illustrates the insertion of the PCR fusion product of FIG. 5 into the HpaI and AatII sites of pFerX4 to generate plasmid pFerX5.

FIG. 7 illustrates the removal of the SwaI site from pFerX5 to generate plasmid pFerX5.1.

FIG. 8 illustrates the addition of the distal 3′ flanking sequences to pFerX6 to generate pFerX7.

FIG. 9 illustrates the addition of the distal 5′ flanking sequences of the ferritin heavy chain gene to pFerX7 to generate plasmid pFerX8.

FIG. 10 illustrates the genetic map of plasmid pFerX8, including the sources of the sequences.

FIG. 11 illustrates the genetic map of plasmid pFerX9, including the sources of the sequences.

FIG. 12 illustrates the sequence of the transcribed region of the pFerX8 and pFerX9 plasmids.

FIG. 13 illustrates the genetic map of pSIDHFR.2, a DHFR expression plasmid.

FIG. 14 shows the results of experiments measuring reporter gene expression in pools of transfectants.

FIG. 15 shows the results of experiments measuring reporter gene expression in transfected isolates.

### DETAILED DESCRIPTION

The patent, scientific and medical publications referred to herein establish knowledge that was available to those of ordinary skill in the art at the time the invention was made. The entire disclosures of the issued U.S. patents, published and pending patent applications, and other references cited herein are hereby incorporated by reference.

Definitions.

All technical and scientific terms used herein, unless otherwise defined below, are intended to have the same meaning as commonly understood by one of ordinary skill in the art; references to techniques employed herein are intended to refer to the techniques as commonly understood in the art, including variations on those techniques or substitutions of equivalent techniques which would be apparent to one of skill in the art. In order to more clearly and concisely describe the subject matter which is the invention, the following definitions are provided for certain terms which are used in the specification and appended claims.

Eukaryotic Locus. As used herein, the term “eukaryotic locus” refers to any chromosomal genetic locus of a eukaryotic cell which encodes a polypeptide or RNA product which can be expressed in the cell under appropriate conditions. Mitochondrial loci are expressly excluded from the scope of the term “eukaryotic locus” as used herein.

Distal 5° Flanking Sequences. As used herein, the term “distal 5′ flanking sequences” refers to flanking nucleotide sequences which are 5′ of the proximal 5′ regulatory sequences of a gene. Thus, although these sequences can have an effect on transcription rates because of their effects on chromatin structure, these sequences are generally 5′ of the basic regulatory sequences (e.g., operators, promoters, ribosome-binding sites) and further removed from the transcriptional initiation site than the proximal 5′ regulatory sequences. The size of the distal 5′ flanking sequences can range between 100-100,000 bases. In certain embodiments, the distal 5′ flanking sequences will include between 500-50,000 bases, 750-25,000 bases or 1,000-10,000 bases. The distal 5′ flanking sequences can begin anywhere 5′ of the proximal 5′ regulatory sequences, and typically begin 20 bases, 50 bases, 75 bases, 100 bases, 500 bases, 1,000 bases, 5,000 bases or 10,000 bases 5′ of the transcription initiation site. Distal 5′ flanking sequences can extend for substantial distances 5′ of the promoter and transcriptional initiation sequences of a gene, and typically end 100,000 bases, 50,000 bases, 25,000 bases or 10,000 bases 5′ of the transcription initiation site.

Proximal 5′ Regulatory Sequences. As used herein, the term “proximal 5′ regulatory sequences” refers to nucleotide sequences which are located near the 5′ end of a gene and which include the basic regulatory elements (i.e., the promoter and, if present, operator and ribosome binding sequences) necessary for transcription and translation. The size of the proximal 5′ regulatory sequences can range between 20-10,000 bases. In certain embodiments, the proximal 5′ regulatory sequences will include between 50-5,000 bases, 75-1,000 bases or 100-500 bases. In some embodiments, the 3′ end of the proximal 5′ regulatory sequences can be defined as immediately .5′ of the translation initiation or “start” codon of the coding region. Alternatively, in some embodiments, the proximal 5′ regulatory sequences can include sequences internal to the gene including intron sequences and, therefore, the 3′ end of the proximal 5′ regulatory sequences can extend to the intron sequences. Moreover, in some embodiments, the proximal 5′ regulatory sequences can include some 5′ coding sequences (e.g., the start codon and/or a short N-terminal sequence). Proximal 5′ regulatory sequences extend 5′ of the transcriptional initiation site, and can end 10,000 bases, 5,000 bases, 1,000 bases, 500 bases, 100 bases, 75 bases, 50 bases or 20 bases 5′ of the transcriptional initiation site.

Proximal 3′ Regulatory Sequences. As used herein, the term “proximal 3′ regulatory sequences” refers to nucleotide sequences which are located near the 3′ end of a gene and which include the basic regulatory elements (i.e., the translational termination codon, polyadenylation signal and transcriptional terminator) necessary for proper MRNA processing and translation termination. The size of the proximal 3′ regulatory sequences can range between 10-2,000 bases. In certain embodiments, the proximal 3′ regulatory sequences will include between 25-1,000 bases, 50-750 bases or 75-500 bases. The 5′ end of the proximal 3′ regulatory sequences can be defined by the translational termination or “stop” codon (i.e., TAG, TTA or TGA). Proximal 3′ regulatory sequences extend 3′ of the translational termination codon, and can end 2,000 bases, 1,000 bases, 750 bases or 500 bases 3′ of the translational termination codon.

Distal 3′ Flanking Sequences. As used herein, the term “distal 3′ flanking sequences” refers to flanking nucleotide sequences which are 3′ of the proximal 3′ regulatory sequences of a gene. Thus, these sequences are 3′ of the basic regulatory sequences (i.e., the stop codon, and polyadenylation signal) necessary for proper mRNA processing and translation termination, and are further removed from the transcriptional termination site than the proximal 3′ regulatory sequences. The size of the distal 3′ flanking sequences can range between 100-100,000 bases. In certain embodiments, the distal 3′ flanking sequences will include between 500-50,000 bases, 750-25,000 bases or 1,000-10,000 bases. The distal 3′ flanking sequences can begin anywhere 3′ of the proximal 3′ regulatory sequences, and typically begin 500 bases, 750 bases, 1,000 bases or 2,000 bases 3′ of the translation termination codon. Distal 3′ flanking sequences can extend for substantial distances 3′ of the transcriptional termination codon and polyadenylation sequences of a gene, and typically end 100,000 bases, 50,000 bases, 25,000 bases or 10,000 bases 3′ of the transcriptional termination codon.

Vector. As used herein, the term “vector” means any genetic construct, such as a plasmid, phage, transposon, cosmid, chromosome, virus, virion, etc., which is capable transferring nucleic acids between cells. Vectors may be capable of one or more of replication, expression, recombination, insertion or integration, but need not possess each of these capabilities. Thus, the term includes cloning and expression vectors.

Transfection. As used herein, the term “transfection” means the introduction into a cell or an organism of a vector that replicates within that cell or organism or that expresses a polypeptide sequence in that cell or organism with or without integrating into the genome of that cell or organism. The term “transfection” is used to embrace all of the various methods of introducing such vectors, including, but not limited to the methods referred to in the art as transfection, transformation, transduction, or gene transfer, and including techniques such as microinjection, DEAE-dextran-mediated endocytosis, calcium phosphate coprecipitation, electroporation, liposome-mediated transfection, ballistic injection, viral-mediated transfection, and the like. Cells or organisms which have undergone transfection are referred to herein as “transfectants.”

Stable Transfection. As used herein, the term “stable transfection” means transfection, as defined above, which results in integration of all or a part of the vector into the genome of the transfected cell or organism. Cells or organisms which have undergone stable transfection are referred to herein as “stable transfectants.”

Operably Joined. As used herein, the term “operably joined” refers to a covalent and functional linkage of genetic regulatory elements and a genetic coding region which can cause the coding region to be transcribed into mRNA by an RNA polymerase which can bind to one or more of the regulatory elements. Thus, a regulatory region, including regulatory elements, is operably joined to a coding region when RNA polymerase is capable under permissive conditions of binding to a promoter within the regulatory region and causing transcription of the coding region into mRNA. In this context, permissive conditions would include standard intracellular conditions for constitutive promoters, standard conditions and the absence of a repressor or the presence of an inducer for repressible/inducible promoters, and appropriate in vitro conditions, as known in the art, for in vitro transcription systems.

Heterologous. As used herein, the term “heterologous” means, with respect to two or more genetic sequences, that the genetic sequences are not operably joined in nature or do not naturally occur within the same genome in nature. For example, if a vector includes a coding region which is operably joined to one or more regulatory elements, these sequences are considered heterologous to each other if they are not operably joined in nature or they are not found in the same genome in nature.

Nucleotide Positions. As used herein, all nucleotide positions are designated with respect to the strand of DNA which includes elements of the ferritin heavy chain gene region in the “sense” orientation. As will be apparent from the context, numerical nucleotide positions are either designated with respect to the position of the start codon of the ferritin heavy chain gene or with respect to the position within one of the sequences included in the Sequence Listing. In the former case, the adenosine or “A” of the start codon (ATG) is designated as position 1, with preceding positions being negatively numbered. In the latter case, the relevant SEQ ID NO will always be specified. Relative nucleotide positions will be described with reference to the conventional 5′ and 3′ directions on the sense strand.

Percentages of Nucleotide Sequence Identity. As used herein, the percentage of sequence identity between two nucleotide sequences are calculated based upon the number of residues which are identical between the aligned sequences divided by the number of nucleotides present in the smaller of the two sequences. Before calculation of the percentage identity, the sequences are aligned using the algorithm (or an equivalent algorithm) of the ClustalW program with default values, available through the European Bioinformatics Institute of the European Molecular Biology Laboratory (EMBL) (http://www.ebi.ac.uk/clustalw), and described in Higgins et al. (1994), “CLUSTAL W: Improving the sensitivity of progressive multiple sequence alignment through sequence weighting, position-specific gap penalties and weight matrix choice,” Nucleic Acids Res. 22:4673-4680.

Derived From. As used herein, the term “derived from,” when used in relation to the origin of a nucleotide sequence, means that the sequence has been or can be obtained or produced, directly or indirectly, from a reference sequence by making a limited number of insertions, deletions or substitutions in the reference sequence. Thus, for example, a sequence which is a subset of a reference sequence can be derived from the reference sequence by deleting flanking sequences. Similarly, a sequence can be derived from a reference sequence by a combination of insertions, deletions and/or substitutions of one or more nucleotides in a reference sequence. The number of insertions, deletions and substitutions can be limited by a required percentage identity between the reference sequence and the derived sequence.

Numerical Ranges. As used herein, the recitation of a numerical range for a variable is intended to convey that the invention may be practiced with the variable equal to any of the values within that range. Thus, for a variable which is inherently discrete, the variable can equal each integer value of the numerical range, including the end-points of the range. Similarly, for a variable which is inherently continuous, the variable can equal each real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value ≦2 for variables which are inherently continuous.

Or. As used herein, unless specifically indicated otherwise, the conjunction “or” is used in the “inclusive” sense of “and/or” and not the “exclusive” sense of “either/or.”

General Considerations.

The present invention depends, in part, upon the development of a high expression “locus vector” derived from the ferritin heavy chain gene. The concept of a “locus vector” is based on the observation that the regions found 5′ and 3′ to highly expressed genes in their natural chromatin contexts can confer higher levels of expression to a heterologous gene. Therefore, the present invention provides a ferritin heavy chain gene locus vector which includes 5′ and 3′ sequences which convey high levels of expression to heterologous genes in stable transfectants. Thus, the invention provides genetic vectors for the stable transfection and expression at high levels of a desired protein within eukaryotic cells.

The Ferritin Heavy Chain Gene.

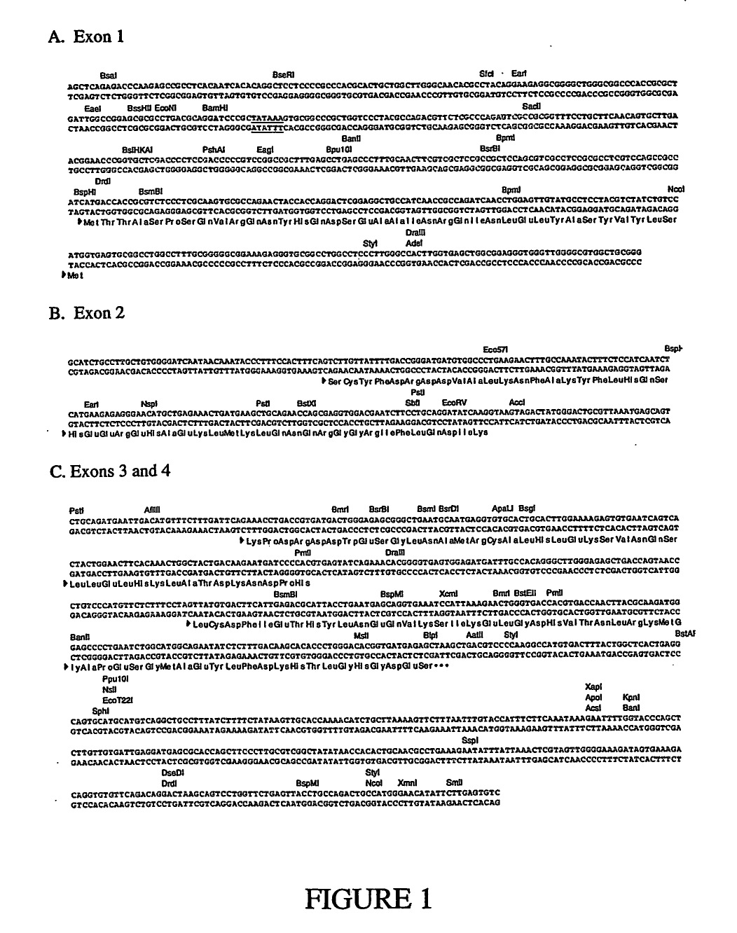

The rat and human genomes contain multiple processed pseudogenes of the ferritin heavy chain (Hentze et al. (1986), Proc. Natl. Acad. Sci. USA 83:7226-72307). The rat ferritin gene consists of four exons (i.e., exons 1 through 4) separated by three introns (i.e., introns 1 through 3). GenBank Accession Nos. M18051, M18052 and M18053 disclose three gene segments which are shown in parts A, B, and C of FIG. 1. Together these three segments cover the four exons of the rat ferritin heavy chain genomic sequence. FIG. 1(A) shows 168 bp of 5′ untranslated sequence, including the transcriptional initiation site at position −168, followed by exon 1 and the first 104 bp of the 5′ end of intron 1. Exon 1 includes the start codon and encodes 38 amino acids. FIG. 1(B) shows the last 50 bp of the 3′ end of intron 1, followed by exon 2 and the first 35 bp of the 5′ end of intron 2. FIG. 1(C) shows the last 33 bp of the 3′ end of intron 2, followed by exon 3, intron 3, exon 4 and 3′ untranslated sequence, including the stop codon and polyadenylation signal 132 bp after the termination codon.

Because the insert sizes of cosmid libraries are quite large, they were chosen to obtain sufficient 5′ and 3′ flanking regions. In particular, rat cosmid library Catalog #RL1032m (13D Biosciences/Clontech, Palo Alto, Calif.) was selected. Other libraries, however, also could have been used, or sequences could have been prepared synthetically.

In order to avoid cloning processed pseudogenes when screening the cosmid library, intron sequences were chosen to serve as probe templates. These introns were cloned by PCR using rat genomic DNA (Catalog #6750-1, Clontech, Palo Alto, Calif.) as a template and primers based on related cDNA and genomic sequences from GenBank. Biotinylated probes were prepared using the introns as templates, and the cosmid library was screened with them. One ferritin heavy chain gene cosmid (15A) was isolated and mapped with restriction enzymes. The three segments of rat genomic sequence from GenBank served as a guide to locate the coding regions and to plan the production of the high expression locus vector.

Production of Ferritin Heavy Chain Gene High Expression Locus Vector.

The production of a high expression locus vector of the invention can be accomplished in many ways. For example, the sequences forming the vector can be obtained from a single clone or from multiple clones. The sequences can be based entirely on the rat ferritin heavy chain gene, entirely on another mammalian ferritin heavy chain, or on multiple mammalian ferritin heavy chain genes. The sequences can be based on all naturally-derived sequences or a mixture of naturally-derived and synthetic sequences. In addition, the locus vector can be produced by first obtaining one or more large genomic fragments including all or part of the ferritin heavy chain gene region and then deleting or inactivating undesired sequences while inserting desired sequences, or can be produced by cloning or subcloning only the desired fragments of the ferritin heavy chain gene region and then combining these with other desired sequences. Similarly, mixtures of these approaches, employing cloning, subcloning, deletion, inactivation and insertion can be employed to arrive at the desired construct. The approach taken and the order of the various steps is irrelevant to the invention and is within the discretion of one skilled in the art.

The high expression locus vectors of the invention include, in order from 5′ to 3′, (a) distal 5′ flanking sequences of a eukaryotic locus; (b) proximal 5′ regulatory sequences of a eukaryotic locus; (c) at least a first insertion site for a heterologous sequence; and (d) proximal 3′ regulatory sequences effective for transcription termination of a eukaryotic locus. Optionally, linker sequences may be present between segments (a)-(d). Furthermore, at least one of the distal 5′ flanking sequences and proximal 5′ regulatory sequences has substantial identity with corresponding sequences of a ferritin heavy chain gene. In some embodiments, distal 3′ flanking sequences are also included in the vector.

One embodiment of a high expression locus vector of the invention, the pFerX8 vector described below, is disclosed in GenBank Accession No. AY147930.

A. Distal 5′ Flanking Sequenced and Proximal 5′ Regulatorv Sequences.

In some embodiments, the distal 5′ flanking sequences of the locus vector will include a sequence of 100-100,000 nucleotides having at least 70%-100% identity to a nucleotide sequence found within the distal 5′ flanking sequences of a ferritin heavy chain locus. Thus, in some embodiments, the distal 5′ flanking sequences can include at least 100, 500, 750, 1,000, 10,000, 25,000, 50,000 or 100,000 nucleotides having at least 70%, 75%, 80%, 85%, 90%, 95% or 100% identity to a nucleotide sequence found within the distal 5′ flanking sequences of a ferritin heavy chain locus. As shown in the examples below, the distal 5′ sequences can include 1,000-10,000 bp, 2,000-9,000 bp, 3,000-8,000 bp or 4,000-7,000 bp of flanking sequences.

In other embodiments, the distal 5′ flanking sequences of the locus vector will share lower percentages identity with the corresponding ferritin heavy chain gene sequences, and in some embodiments the distal 5′ flanking sequences will be unrelated to any corresponding ferritin heavy chain gene sequences.

Downstream from the distal 5′ flanking sequences, the high expression locus vector of the invention includes proximal 5′ regulatory sequences. In some embodiments, the proximal 5′ regulatory sequences of the locus vector will include a sequence of at least 20-10,000 nucleotides having at least 70%-100% identity to a nucleotide sequence found within the proximal 5′ regulatory sequences of a ferritin heavy chain locus. Thus, in some embodiments, the proximal 5′ regulatory sequences can include at least 20, 50, 75, 100, 500, 1,000, 5,000 or 10,000 nucleotides having at least 70%, 75%, 80%, 85%, 90%, 95% or 100% identity to a nucleotide sequence found within the proximal 5′ regulatory sequences of a ferritin heavy chain locus.

In other embodiments, the proximal 5′ regulatory sequences of the locus vector will share lower percentages identity with the corresponding ferritin heavy chain gene sequences, and in some embodiments the proximal 5′ regulatory sequences will be unrelated to any corresponding ferritin heavy chain gene sequences.

In all embodiments, the proximal 5′ regulatory sequences must be effective to initiation transcription of the heterologous coding region to be inserted into the vector. Thus, in those embodiments in which the proximal 5′ regulatory sequences are based upon the corresponding ferritin heavy chain gene sequences, they should not be varied to such an extent that the sequences become ineffective in initiating and promoting transcription. Thus, the conservation of features such as the “TATA box” or ribosome binding site, or the replacement of these features with equivalent sequences, is necessary to preserve functionality of the expression vector. On the other hand, it is also acceptable to completely replace these sequences with functional equivalents from other genes, including any of the many known proximal 5′ regulatory regions from other genes. Similarly, it is acceptable to replace these sequences with chimeric sequences based upon the proximal 5′ regulatory regions of two or more genes.

In some embodiments, both the distal 5′ flanking sequences and the proximal 5′ regulatory sequences include a sequence of at least 100-1000 nucleotides having at least 70%-100% identity to a nucleotide sequence found within, respectively, the distal 5′ flanking and proximal 5′ regulatory sequences of a ferritin heavy chain locus. In some of these embodiments, the distal 5′ flanking sequences and the proximal 5′ regulatory sequences have 70-100% identity to contiguous sequences found within a ferritin heavy chain locus.

Because intron 1 of the ferritin heavy chain gene can contain positive regulatory elements, and can aid in RNA processing and transport, it can be advantageous to create a locus vector that includes the maintenance of all or a portion of intron 1 as part of the proximal 5′ regulatory sequences. This can be accomplished by maintaining an ATG codon and, optionally, additional codons 5′ to the beginning of the intron 1 sequences. If codons other than the ATG are maintained, they can be derived from the ferritin heavy chain gene exon 1 coding sequences or any other coding sequences (including synthetic or artificial sequences), and will encode the N-terminus of a fusion protein with the heterologous coding sequences. Such an N-terminus can function as a leader or signal sequence to aid in expression of the heterologous sequences. Alternatively, in other embodiments, an additional heterologous sequence insertion site (e.g., a single restriction site or a polylinker) can be inserted 5′ to the beginning of intron 1 so that sequences encoding various N-terminal sequences (e.g., leader or signal sequences) can be inserted at will. The ATG codon can be provided as part of the vector, or can be part of the inserted heterologous sequences.

However, there is no need to maintain either the ATG codon or any other codons prior to intron 1. Rather, in some embodiments, the ATG codon can be present in exon 2 or can be provided by a heterologous coding sequence. In such embodiments, the heterologous sequence insertion site will be present in exon 2, or at the intron 1/exon 2 junction, and the ATG codon either can be provided as part of the vector, or can be part of the inserted heterologous sequences. In all instances in which intron 1 is included in the vector, however, the splice donor and splice acceptor sequences of intron 1, or equivalent splice donor and acceptor sequences, must be maintained so that the intron sequences are post-transcriptionally removed. Other sequences within the intron can be deleted or varied, or additional sequences can be inserted, as described herein. However, in constructs in which intron 1 is maintained, insertion of a heterologous coding region, whether 5′ or 3′ of intron 1, must not disrupt the splice donor and acceptor sites, must reconstruct the splice donor and acceptor sites, or must provide equivalent splice donor and acceptor sites.

Finally, because the ferritin heavy chain gene exon 1 also contains an iron regulatory element (IRE) 3′ to the ATG (at approximately positions −138 to −111) that negatively controls translation depending on the level of iron (reviewed in Klausner et al. (1993), Cell 72:19-26), the creation of the locus vector can optionally include the deletion of the IRE from the proximal 5′ regulatory sequences.

B. Ferritin Heavy Chain Coding Regions.

Typically, the locus vector will not include any coding regions from the ferritin heavy chain gene. However, depending upon the method by which the vector is created, ferritin heavy chain coding regions can be included intentionally or as artifacts. For example, if the entire ferritin heavy chain gene region is cloned into a vector with the intention of using only the distal 5′ flanking sequences and/or proximal 5′ regulatory sequences (together “the 5′ ferritin sequences”), the coding regions can be purposefully deleted in their entirety. Alternatively, a heterologous sequence insertion site (e.g., a single restriction site or a polylinker) and proximal 3′ regulatory sequences (and optionally distal 3′ flanking sequences) could be inserted immediately 3′ to the 5′ ferritin sequences without deleting the coding regions. Because of the intervening insertion, the coding regions would be inactivated. Similarly, all of the coding regions except the start codon could be deleted or, alternatively, the heterologous sequence insertion site and proximal 3′ regulatory sequences (and optionally distal 3′ flanking sequences) could be inserted immediately 3′ to the start codon. In addition, a larger portion of the coding region can be maintained before the insertion of the heterologous sequence insertion site and proximal 3′ regulatory sequences (and optionally distal 3′ flanking sequences) so that a fusion protein can be produced. Finally, combinations of the foregoing approaches can be employed such that the ferritin heavy chain coding regions are partially deleted and partially inactivated by the insertion of intervening sequences. In some embodiments, however, in order to reduce the size of the vector, inactivated and untranslated sequences are deleted.

C. Heterologous Sequence Insertion Site.

Downstream from the proximal 5′ regulatory sequences, the high expression locus vector of the invention includes an insertion site for a heterologous sequence, such as a polylinker site. The heterologous sequence insertion site can be any sequence into which a heterologous sequence can be inserted in a sufficiently controlled and predictable manner to allow for production of functional high expression locus vectors with a reasonable expectation of success. Insertion sites for a heterologous sequence can include sites for homologous recombination, site-directed integration (e.g., via transposons or viral constructs), or endonuclease-mediated restriction. The length of the insertion site can vary from 4 bp (for use with four-cutter restriction endonucleases) to 1,000 bp or 5,000 bp (for use with homologous recombination methods). However, in certain circumstances, the 3′ end of the proximal 5′ regulatory sequences and the 5′ end of the proximal 3′ regulatory sequences can form an insertion site without the need for the inclusion of additional nucleotides between them. Thus, for example, the last two nucleotides of the proximal 5′ regulatory sequences and the first two nucleotides of the proximal 3′ regulatory sequences can form a 4 bp restriction site which can serve as an insertion site for the heterologous sequences. Alternatively, only one or a few nucleotides may be required to form an insertion site between these sequences. Thus, the length of the insertion site could be 0, 1, 2, or 3 bp, as well as the 4 bp to 5,000 bp described above.

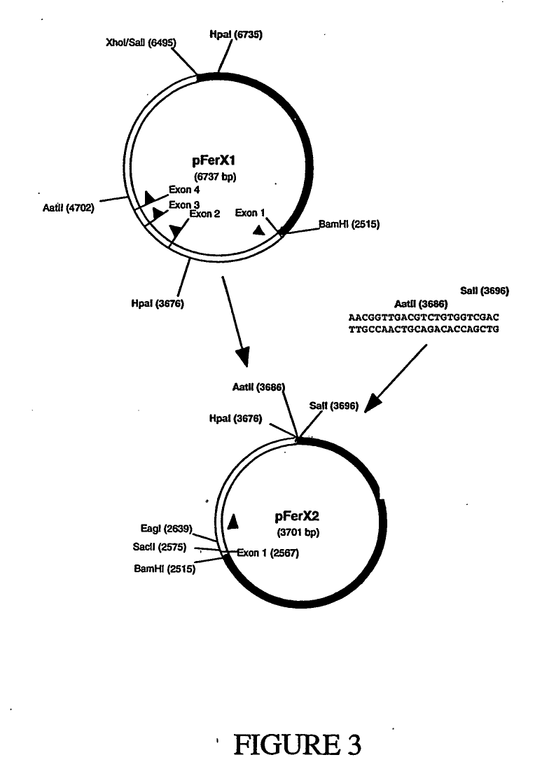

In some embodiments, the heterologous sequence insertion site will include one or more nucleotide sequences, on either the sense or antisense stand, which serve as restriction site(s) for natural or artificial endonucleases. These restriction sites can be unique in the vector, and the insertion site can be a polylinker that includes a multiplicity of such restriction sites to afford greater flexibility of use with different restriction endonucleases. An example of such a polylinker is provided in Example 1 and FIG. 3.

D. Proximal 3′ Regulatory Sequences.

Downstream from the insertion site for the heterologous sequences, the high expression locus vector of the invention includes proximal 3′ regulatory sequences. At a minimum, these sequences include a polyadenylation signal. In some embodiments, the proximal 3′ regulatory sequences also include a transcriptional termination signal. In some embodiments, the sequences can include the translation termination or stop codon, whereas in other embodiments the stop codon will be included in the heterologous sequence insert.

The proximal 3′ regulatory sequences can be derived from the ferritin heavy chain gene, but need not be. For example, in some embodiments, the proximal 3′ regulatory sequences of the locus vector will include a sequence of at least 10-2,000 bases nucleotides having at least 70%-100% identity to a nucleotide sequence found within the proximal 3′ flanking sequences of a ferritin heavy chain locus. Thus, in some embodiments, the proximal 3′ regulatory sequences can include at least 10, 25, 50, 100, 500, 750, 1,000, or 2,000 nucleotides having at least 70%, 75%, 80%, 85%, 90%, 95% or 100% identity to a nucleotide sequence found within the proximal 3′ regulatory sequences of a ferritin heavy chain locus. In other embodiments, the proximal 3′ regulatory sequences will consist essentially of a polyadenylation signal, which can be derived from a ferritin heavy chain gene, a heterologous sequence, or a synthetic or artificial sequence.

In other embodiments, the proximal 3′ regulatory sequences of the locus vector will share lower percentages identity with the corresponding ferritin heavy chain gene sequences, and in some embodiments the proximal 3′ regulatory sequences will be unrelated to any corresponding ferritin heavy chain gene sequences.

E. Distal 3′ Flanking Sequences.

Downstream from the proximal 3′ regulatory sequences, the high expression locus vector of the invention optionally includes distal 3′ flanking sequences. The distal 3′ flanking sequences can be derived from the ferritin heavy chain gene, but need not be. For example, in some embodiments, the distal 3′ flanking sequences of the locus vector will include a sequence of at least 100-100,000 nucleotides having at least 70%-100% identity to a nucleotide sequence found within the distal 3′ flanking sequences of a ferritin heavy chain locus. Thus, in some embodiments, the distal 3′ flanking sequences can include at least 100, 500, 750, 1,000, 10,000, 25,000, 50,000, or 100,000 nucleotides having at least 70%, 75%, 80%, 85%, 90%, 95% or 100% identity to a nucleotide sequence found within the distal 3′ flanking sequences of a ferritin heavy chain locus. As shown in the examples below, the distal 3′ flanking sequences can include 1,000-10,000 bp, 2,000-9,000 bp, 3,000-8,000 bp or 4,000-7,000 bp of flanking sequences.

In other embodiments, the distal 3′ flanking sequences of the locus vector will share lower percentages identity with the corresponding ferritin heavy chain gene sequences, and in some embodiments the distal 3′ flanking sequences will be unrelated to any corresponding ferritin heavy chain gene sequences.

The following examples illustrate some specific modes of practicing the present invention, but are not intended to limit the scope of the claimed invention. Alternative materials and methods may be utilized to obtain similar results.

### EXAMPLE 1

Creation of a Ferritin Heavy Chain Locus Vector.

In order to generate a high expression locus vector based on the ferritin heavy chain gene, three phases of development were employed: (1) cloning of a ferritin heavy chain gene with substantial 5′ and 3′ regions; (2) production of an expression vector based on at least one of these gene regions, and (3) optimization of the vector. As noted above, many other approaches could have been employed to produce the same or equivalent locus vectors.

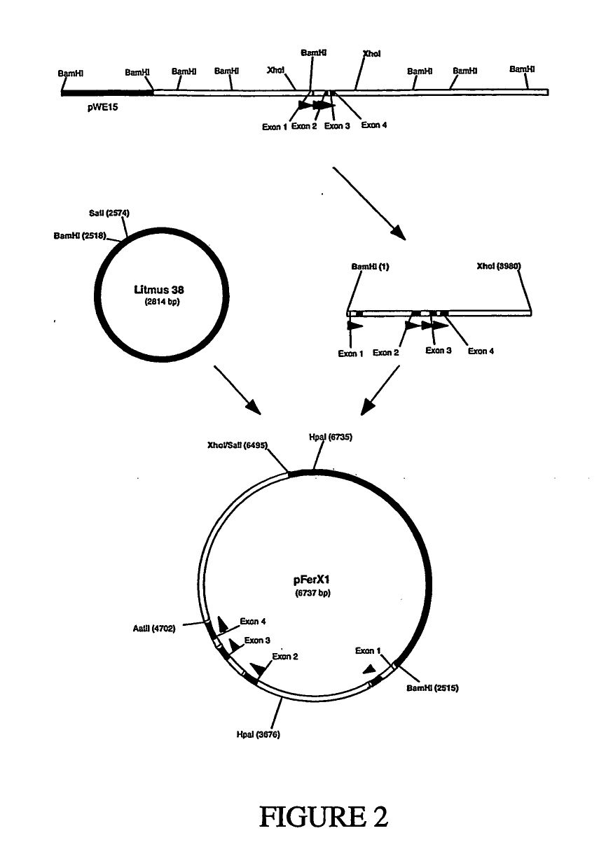

First, the region containing the ferritin heavy chain exons from cosmid 15A was subcloned into the Litmus 38 vector (New England Biolabs) to generate plasmid pFerX1 (FIG. 2). The BamHI-XhoI fragment was isolated from cosmid 15A and ligated into Litmus 38 digested with BamHI and SalI to generate plasmid pFerX1. Note that cosmid 15A was only partially sequenced and that some of the restriction site locations are based on restriction mapping. Therefore, some of the restriction site locations may not be accurate.

FIG. 3 illustrates the deletion of the fragment containing exons 2, 3, and 4 from pFerX1 and the insertion of a polylinker containing AatII and SalI restriction sites to generate plasmid pFerX2. The deleted Hpal fragment extended from the HpaI site in the insert to the HpaI site in the vector in pFerX1. The 5′ end of the polylinker regenerated the HpaI site, but the 3′ end did not. Screening for the orientation of the linker was done using PCR.

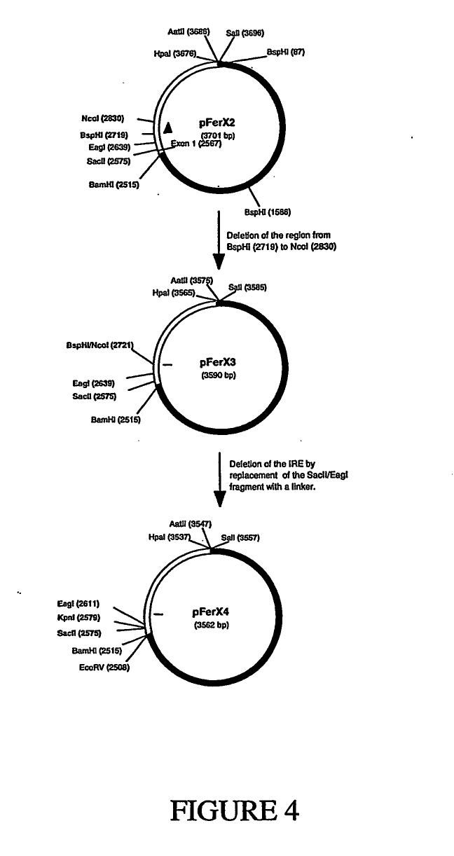

The exon 1 coding region was deleted from pFerX2, leaving the ATG initiation codon and the following splice donor intact to generate plasmid pFerX3. FIG. 4 illustrates that the deletion of the exon 1 coding region was accomplished by isolating the BamHI-BspHI (2515-2719) and NcoI-BamHI (2830-2515) fragments from pFerX2. BspHI and NcoI generate compatible overhangs which permitted the resulting fragments to be ligated together to generate pFerX3. As a result of this manipulation, exon 1 of the vector was changed from:

```
                    BspHI
CCAGCCGCCATC ATG ACC ACC GCG TCT CCC TCG CAA GTG CGC CAG AAC TAC CAC CAG GAC TCG GAG GCT
GGTCGGCGGTAG TAC TGG TGG CGC AGA GGG AGC GTT CAC GCG GTC TTG ATG GTG GTC CTG AGC CTC CGA [symbol]
                      Met Thr Thr Ala Ser Pro Set Gln Val Arg Gln Asn Tyr His Gln Asp Set Glu Ala
                                                                                                                                          NcoI Splice Donor
GCC ATC AAC CGC CAG ATC AAC CTG GAG TTG TAT GCC TCC TAC GTC TAT CTG TCC ATG GTGAGTGCGGCCT
CGG TAG TTG GCG GTC TAG TTG GAC CTC AAC ATA CGG AGG ATG CAG ATA GAC AGG TAC CACTCACGCCGGA [symbol]
Ala Ile Asn Arg Gln Ile Asn Leu Glu Leu Tyr Ala Ser Tyr Val Tyr Leu Set Met
```

```
                                  Splice Donor
CCAGCCGCCATC ATG GTGAGTGCGGCCT
GGTCGGCGGTAG TAC CACTCACGCCGGA [symbol]
                        Met
```

Deletion of the exon 1 IRE was accomplished by replacing the SacII-EagI (2575-2639) fragment in pFerX3 with a linker that does not contain the IRE (but creates a 5′ KpnI site for screening) to generate plasmid pFerX4. As a result of this manipulation, exon 1 of the vector was changed from (IRE underlined):

SacII (2575) EagI (2639)
CAGAGTCGCCGCGGTTTCCTGCTTCAACAGTGCTTGAACGGAACCCGGTGCTCGACCCCTCCGACCCCCGTCCGGCCGCTTTGAGCC
GTCTCAGCGGCGCCAAAGGACGAAGTTGTCACGAACTTGCCTTGGGCCACGAGCTGGGGAGGCTGGGGGCAGGCCGGCGAAACTCGG

to (linker shown in bold):

```
                        KpnI (2579)
                SacII (2575) EagI (2611)
CAGAGTCGCCGCGGTACCGGTGCTCGACCCCTCCGACCCCCGTCCGGCCGCTTTGAGCC
GTCTCAGCGGCGCCATGGCCACGAGCTGGGGAGGCTGGGGGCAGGCCGGCGAAACTCGG
```

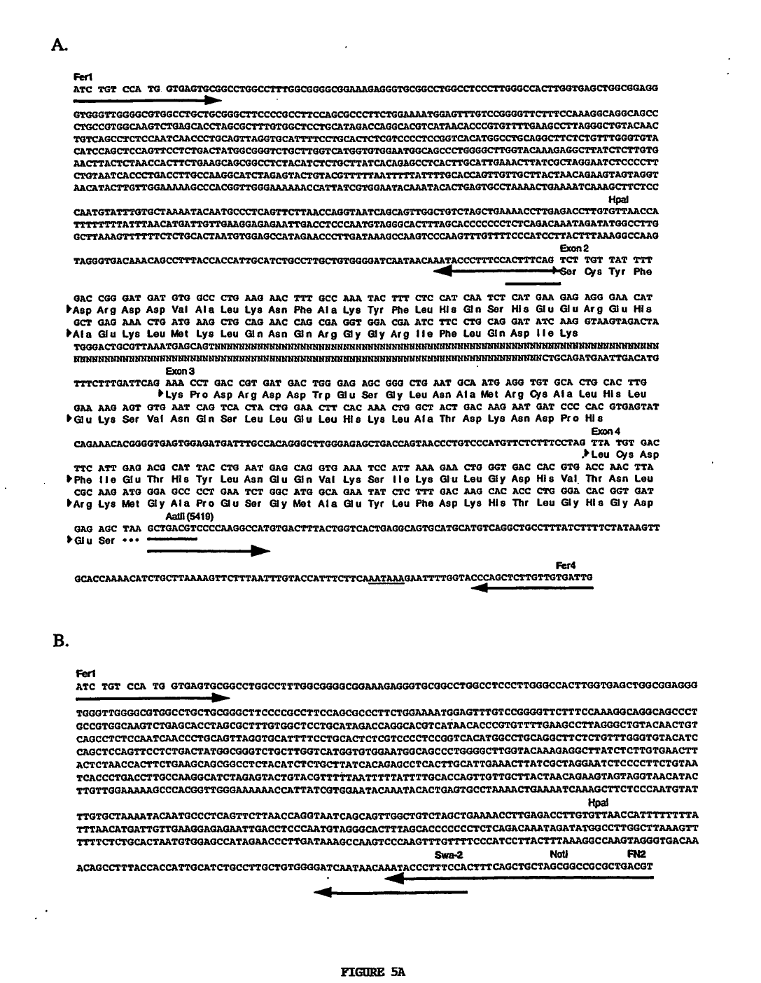

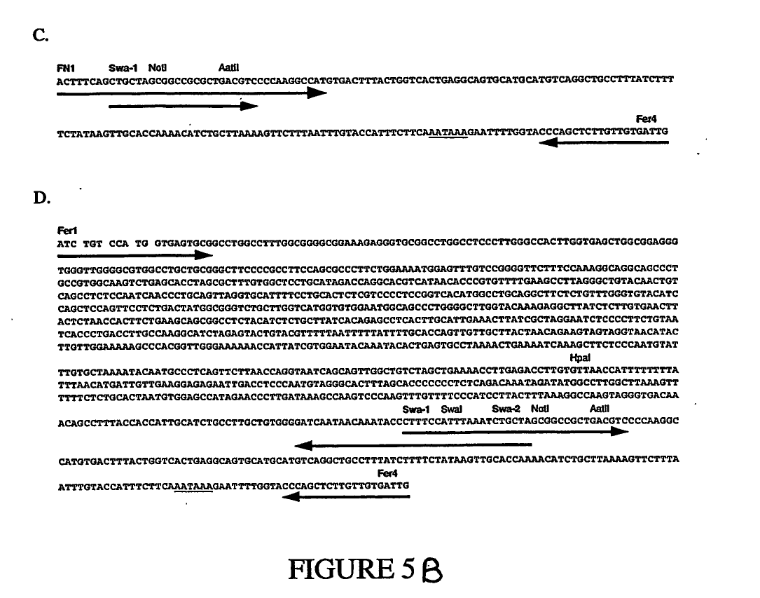

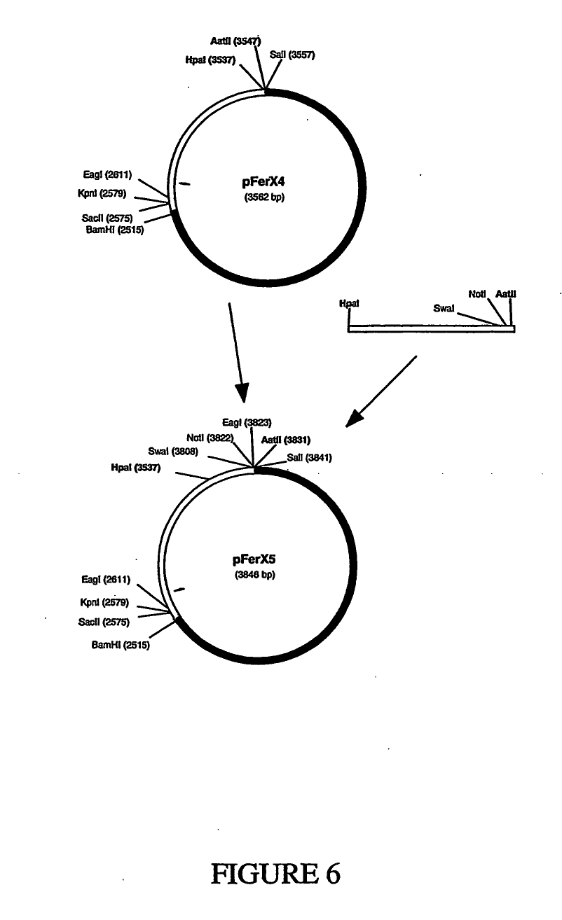

A PCR fusion product was generated in a three step procedure to replace exons 2 though 4 with a polylinker containing SwaI and NotI, while maintaining the proximal 5′ regulatory sequences and proximal 3′ regulatory sequences of the ferritin heavy chain gene. As shown in FIG. 5(A), the first PCR used cosmid 15A (FIG. 2) as a template. Primer locations for primers Fer1 and Fer4 are indicated by arrows. The “priming” region for primers FN1 and FN2 are also indicated by bars. In the second step, shown in FIG. 5(B), a Fer1-FN2 PCR product was generated. The location of the “priming” region of primer Swa-2 is indicated. In the third step, shown in FIG. 5(C), a FN1-Fer4 PCR product was generated. The location of the “priming” region of primer Swa-1 is indicated. In the fourth and final step, as shown in FIG. 5(D), the final PCR fusion product was generated by using the Fer1-Swa-2 and Swa-1-Fer4 products as templates and the Fer1 and Fer4 primers. The HpaI-AatII fragment was isolated from this product for insertion into the HpaI and AatII sites of pFerX4 to generate plasmid pFerX5 (see FIG. 6). The PCR fusion reactions used in the first three steps to generate the SwaI-NotI polylinker are shown in TABLE 1.

TABLE 1

| | Template(s) | 5′ primer | 3′ primer |
|---|---|---|---|
| First PCR | Cosmid 15A | Fer1 | FN2 |
| | Cosmid 15A | FN1 | Fer4 |
| Second PCR | Fer1/FN2 | Fer1 | Swa-2 |
| | product | | |
| | FN1/Fer4 | Swa-1 | Fer4 |
| | product | | |
| Third PCR | Fer1/Swa-2 & | Fer1 or Fer3 | Fer4 |
| | Swa-1/Fer4 | | |
| | products | | |

The PCR primers are shown below, where the polylinker sequence is shown in bold, and the complementary sequences between FN1 and FN2 or between Swa-1 and Swa-2 are shown underlined.

| | |
|---|---|
| | NheI NotI AatTI |
| FN1 | ACTTTCAGCTGCTAGCGGCCGCGCTGACGTCCCCAAGGCCAT |
| | NotI NheI |
| FN2 | ACGTCAGCGCGGCCGCTAGCAGCTGAAAGTGGAAAGGGTAT |
| | SwaI NotI AatII |
| Swa-1 | CTTTCCATTTAAATCTGCTAGCGGCCGCTGACGTC |
| | SwaI |
| Swa-2 | TAGCAGATTTAAATGGAAAGGGTATTTGTTATTGATC |

The SwaI site in the vector backbone of pFerX4 was removed by blunt cleavage of the plasmid with SwaI and insertion of the double-stranded oligo:

GGCGCGCC
CCGCGCGG

which contains an AscI site to generate plasmid pFerX4.1. The SwaI site was removed from the vector backbone in order to make the SwaI site in the polylinker above unique.

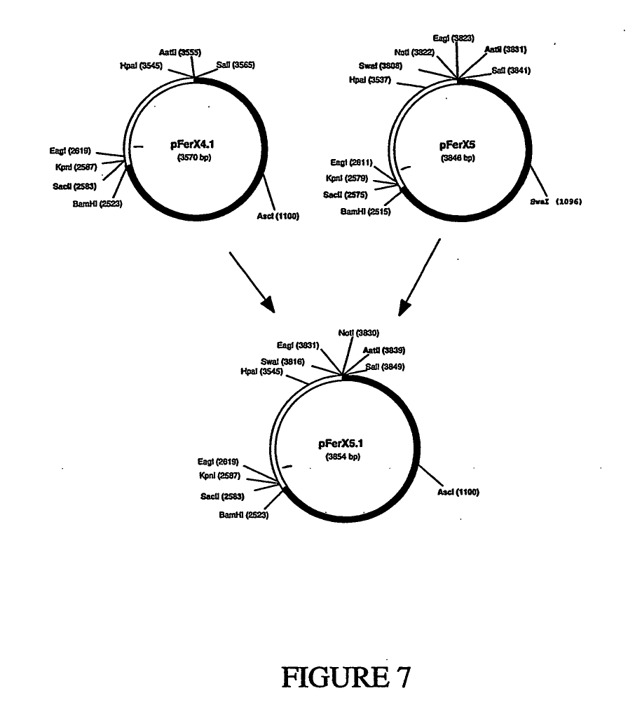

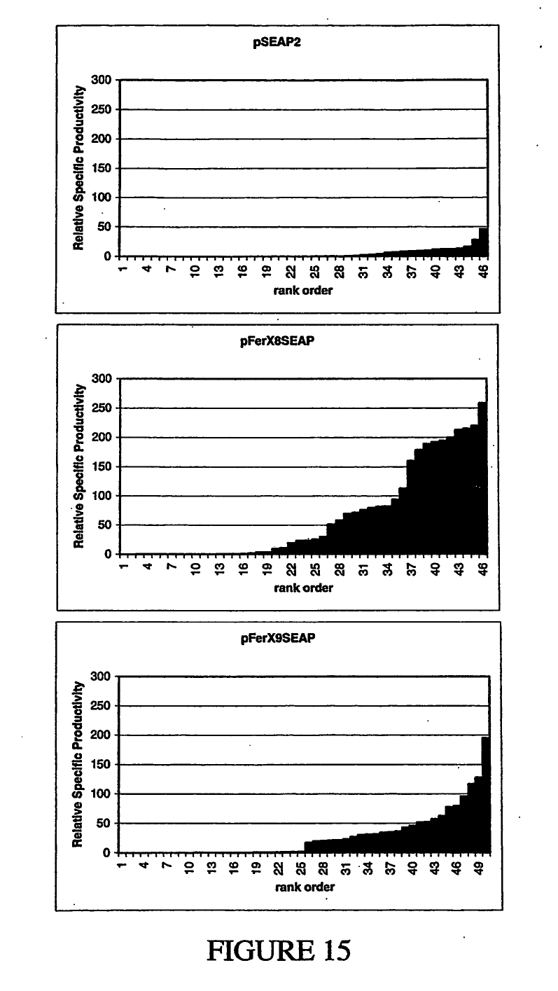

The vector backbones of pFerX4.1 (in which the insertion of the AscI oligo of FIG. 15 destroyed the SwaI site in the vector backbone) and pFerX5 (which included the SwaI site in the backbone and the polylinker) were swapped using SacII-AatII fragments to generate plasmid pFerX5.1 (FIG. 7). pFerX5.1 contained the polylinker and but lacked the SwaI site in the backbone, making the SwaI site in the polylinker unique.

The polylinker

```
            BgIII BstBI
        CTGTGAGATCTGTTCGAATGG
  TGCAGACACTCTAGACAAGCTTACCAGCT
AatII SalI
compatible compatible
```

was inserted into the SalI-AatII sites of pFerX5.1 to generate plasmid pFerX6. The polylinker includes both BglII and BstBI sites and was designed to receive the distal 3′ flanking sequences of the ferritin heavy chain gene.

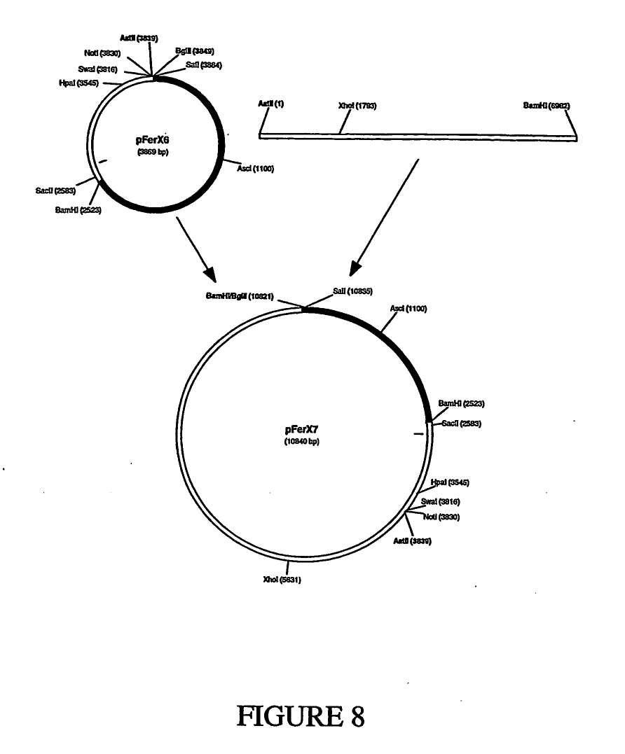

The distal 3′ flanking sequences of the ferritin heavy chain gene (AatII-BamHI fragment from cosmid 15A) were inserted into the AatII-BglII sites of pFerX6 to generate plasmid pFerX7 (FIG. 8).

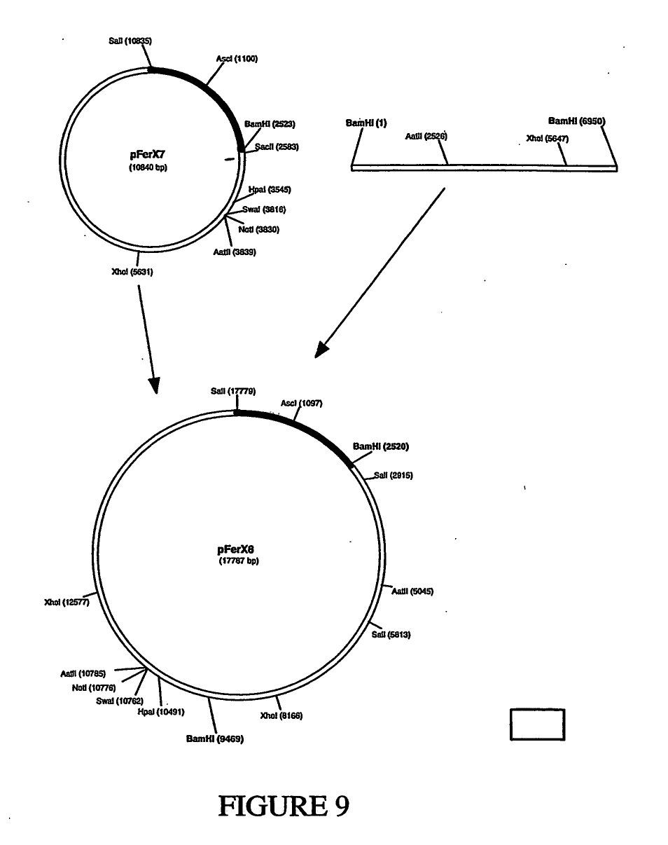

The distal 5′ flanking sequences of the ferritin heavy chain gene (BamHI fragment from pFerH1, a subclone of cosmid 15A, FIG. 2: BamHI 10269-15176) were inserted into the BamHI site of pFerX7 to generate plasmid pFerX8 (FIG. 9).

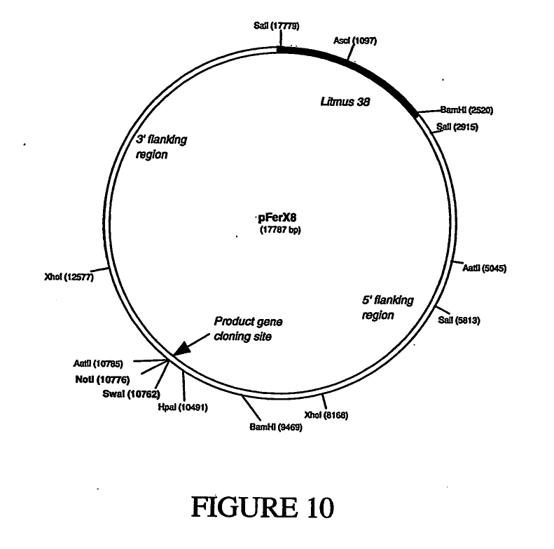

The origins of the various sequences forming pFerX8 are shown in FIG. 10. The Litmus 38 backbone is indicated by the filled box. This plasmid contains >6kb of distal 5′ flanking sequences before the initiating ATG codon and 7 kb of distal 3′ flanking sequences following the termination codon. The SwaI and NotI cloning sites are located at positions 10240 and 10254, respectively. Coding regions inserted into the SwaI and NotI sites should be blunt ended at the 5′ end (SwaI end) and should start with the bases CAG to regenerate the splice acceptor followed by the second amino acid. The NotI site should be present at the 3′ end following the termination codon.

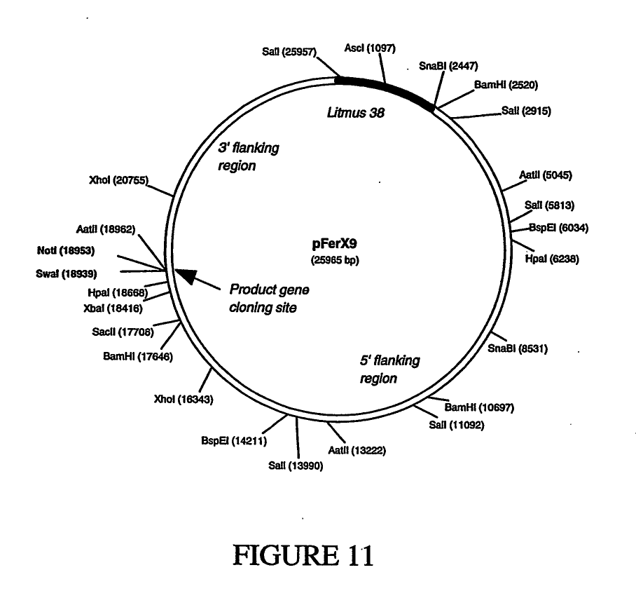

An additional segment of distal 5′ flanking sequence (BspEI fragment from cosmid 15A) was inserted into the BspEI site (6037) of pFerX8 to generate plasmid pFerX9 (FIG. 11; BspE1 fragment 6034-14211). This insertion adds both a unique segment of distal 5′ flanking sequence as well as repeating a segment of the distal 5′ flanking sequence already present in pFerX8 (10697-13990 is the same as 2520-5813). This plasmid is not entirely sequenced and the locations of some of the restriction sites are estimated based on restriction fragment sizes.

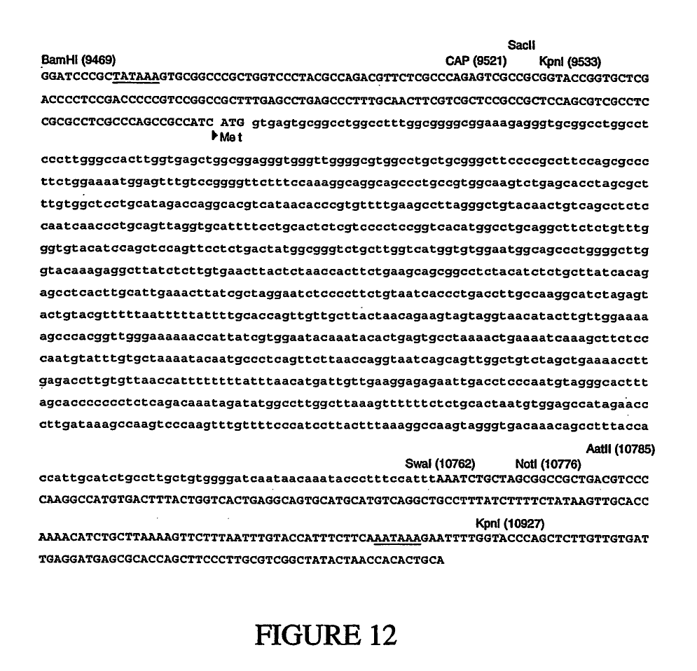

The sequence of the transcribed region of the pFerX8 and pFerX9 plasmids is shown in FIG. 12. The putative transcription start site is indicated. The intron is shown in lower case. The putative TATA and polyadenylation signals are underlined. The initiation codon is in the first exon and the inserted gene, starting with the second amino acid, is inserted into the SwaI and NotI sites.

### EXAMPLE 2

Expression of Heterologous Sequences.

A. Reoorter Gene

A reporter gene was inserted into the SwaI-NotI sites in the polylinker of both the pFerX8 and pFerX9 plasmids. Secreted alkaline phosphatase (SEAP) was selected as a reporter gene because the commercially available assay (Clontech, Palo Alto, Calif.) for the product is simple and rapid. The expression vectors were designated pFerX8SEAP and pFerX9SEAP.

The sequence of the vector polylinker and the original sequence at the 5′ end of exon 2 that needs to be recreated to regenerate the splice donor are shown in FIG. 5. Thus, the 5′ primer should include a CAG at the 5′ end to recreate the natural 20 splice donor followed by the coding region starting with the second amino acid (the ATG is already included in exon 1). The 5′ end of the PCR product should be left blunt-ended for ligation with the SwaI site. For example:

General 5′ primer:

CAG NNN NNN NNN NNN NNN NNN NNN
AA2 AA3 AA4 AA5 AA6 AA7 AA8

Primer for SEAP example:

CAG CTG CTG CTG CTG CTG CTG CTG GGC

The 3′ primer should include a NotI site followed by the 3′ end of the gene including the termination codon (opposite strand). The PCR product should be digested with NotI to generate an end compatible with the NotI site in the polylinker. For example:

General 5′ primer:

```
NNNN GCGGCCGC NNN NNN NNN NNN NNN NNN NNN
          NotI 3′ end of gene
          site
```

Primer for SEAP example (termination codon in bold):

TTTT GCGGCCGC AGC TCA TGT CTG CTC GAA GCG GCC

Ligation of the PCR product with the vector (digested with SwaI and NotI) does not recreate a SwaI site at the 5′ end of the insert. Instead the ligated product contains a suitable splice acceptor at the “SwaI end.” The inserted region will also contain the coding sequence from the second amino acid to the termination codon followed by the NotI site at the 3′ end. For example:

After ligation generally:

CCATTT CAG NNN NNN NNN // NNN NNN NNN GCGGCCGC
TGACGT

Example for SEAP:

CCATTT CAG CTG CTG CTG // CAG ACA TGA GCGGCCGC
TGACGT

B. Transfections

The host used for transfections was the CHO DG44(E) cell line (Urlaub et al. (1986), Somatic Cell Mol. Gen. 12:555-566), which had been selected for growth and survival in serum-free media. This cell line was maintained in a spinner flask in serum-free media with added nucleosides. The cells used for transfection were in exponential growth. Either 2×10⁶ or 5×10⁶ cells were used for each transfection.

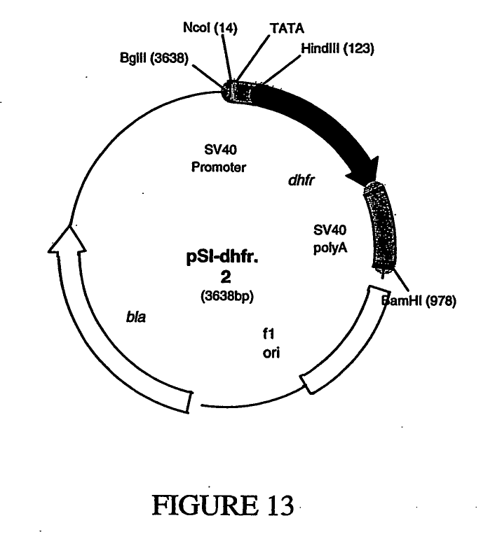

Reporter plasmids were co-transfected with a plasmid designated pSI-DHFR.2 encoding dihydrofolate reductase (DHFR) so that stable transfectants could be selected in the DHFR-host. The pSI-DHFR.2 plasmid includes a selectable marker and the dhfr gene driven by the SV40 promoter with the SV40 enhancer deleted (FIG. 13).

All DNA was prepared by Megaprep kit (Qiagen, Valencia Calif.). Prior to transfection DNA was EtOH precipitated, 70% EtOH washed, dried, resuspended in HEBS (20 mM Hepes pH 7.05, 137 mM NaCl, 5 mM KCl, 0.7 mM Na₂HPO₄, 6 mM dextrose), and quantitated prior to transfection. As a positive control, a plasmid which expresses SEAP with an SV40 early promoter/enhancer (pSEAP2, Clontech, Palo Alto, Calif.) was employed. Negative controls included an empty pUC18 vector (ATCC #37253, American Type Culture Collection, Manasssas, Va.) as a reporter control and a no DNA transfection as a tansfection control.

Each transfection contained 50 μg of a reporter plasmid and 5 μg pSI-DHFR.2. Equal plasmid weight was selected rather than equimolar amounts. From a molarity perspective there are differences on the order of 3-5 fold between the control reporters and the test reporters (TABLE 3). In each case the test reporter was lower than the control.

TABLE 3

| Reporter plasmid | Plasmid | Molar ratio | DHFR | Reporter |
|---|---|---|---|---|
| (50 μg each) | size (kb) | to controls | (5 ug each) | gene |
| pSEAP2 | 5.1 | 1 | pSIDHFR.2 | SEAP |
| pFerX8SEAP | 18.9 | 0.27 | pSIDHFR.2 | SEAP |
| pFerX9SEAP | 26.6 | 0.19 | pSIDHFR.2 | SEAP |
| pUC18 | 2.7 | | pSIDHFR.2 | none |
| No DNA | | | No DNA | none |

Cells and DNA were transfected by electroporation in 0.8 ml of HEBS using a 0.4 cm cuvette (13ioRad, Hercules, Calif.) at 0.28 kV and 950 μF. After the electroporation pulse, the cells were allowed to incubate in the cuvette for 5-10 min at room temperature. They were then transferred to a centrifuge tube containing 10 ml of Alpha-MEM plus nucleosides (GIBCO, Gaithersburg, Md.) with 10% dFBS (HyClone, Logan, Utah) and pelleted at 1K rpm for 5 min. Resuspended pellets were seeded into T-flasks in Alpha-MEM without nucleosides with 10% dFBS and incubated at 36° C. with 5% CO₂ in a humidified incubator until colonies formed.

TABLE 4 summaries seven experiments which were conducted. Transfections 1-3 were each performed in triplicate, and transfections 4-7 were performed once each.

TABLE 4

| | Reporter plasmid | DHFR | Reporter |
|---|---|---|---|
| Exp. # | (50 μg each) | (5 μg each) | gene |
| 1 | pSEAP2 | pSIDHFR.2 | SEAP |
| 2 | pFerX8SEAP | pSIDHFR.2 | SEAP |
| 3 | pFerX9SEAP | pSIDHFR.2 | SEAP |
| 4 | pFerX8 | pSIDHFR.2 | none |
| 5 | pFerX9 | pSIDHFR.2 | none |
| 6 | pUC18 | pSIDHFR.2 | none |
| 7 | No DNA | No DNA | none |

C. Transfection Efficiency

Approximately 2 weeks after the transfections, colonies had formed. Stable transfectants were analyzed as either pools or isolates. Although all the pSI-DHFR.2-containing transfections produced colonies, the transfections containing the ferritin heavy chain locus vectors produced fewer colonies than did the controls. This was true whether or not the locus vector expressed a product. These results were surprising since the same amount of DNA was included in each transfection. Because of the difference in transfection efficiency it is recommended that multiple transfections be done to account for the reduced number of transfectants.

D. SEAP Assay

The reporter constructs containing the SEAP gene were analyzed using the Great EscAPe™ SEAP Reporter System 3 (Clontech, Palo Alto, Calif.). This assay uses a fluorescent substrate to detect the SEAP activity in the conditioned media. The kit was used in a 96-well format according to the manufacturer's instructions with the following exceptions. All standards and samples were diluted in fresh media rather than the dilution buffer provided. Instead of performing one reading after 60 min, multiple reads were taken at 10-20 min intervals and used to express SEAP activity as relative fluorescent units per minute (RFU/min). The emission filter available for the Cytofluor II plate reader was 460 nm instead of the recommended 449 μm.

All of the data generated for the pools and isolates below was based on the reporter constructs expressing SEAP. The titers reported were based on a positive control with the kit. Although absolute values were not derived, the relative titer values are useful.

The specific productivity was assessed in assays in which the media was exchanged for fresh media and then, 24 hours later, the media was sampled and the cells were counted. The product titer was normalized for the cell number at the end of the 24 hour assay. Because the titers were relative, the specific productivities are expressed as relative values.

$$Specific productivity = \frac{product titer ( / ml ) \times volume ( ml )}{time ( days ) \times \# of cells}$$

E. Transfectant Pools

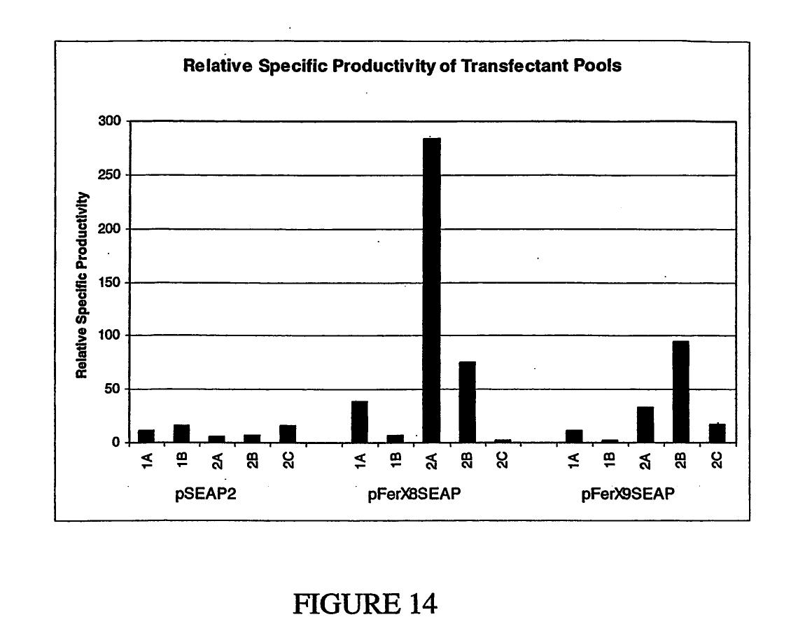

After the appearance of colonies, the cells were collected and pooled from each transfection. Pools were seeded into 6-well plates or T-flasks and were kept subconfluent for the 24 hour assay. Results from the pool assays are shown in FIG. 14. Five pools were analyzed for each construct, two from experiment 1 (1A and 1B) and three from experiment 2 (2A, 2B, and 2C). All assays were done three to four weeks post-transfection. Note that the experiment 2C with pFerX8SEAP had a very low transfection efficiency relative to the other transfections.

Specific productivities were fairly consistent with the control (pSEAP2) but highly variable with the pFerX8SEAP and pFerX9SEAP vectors. Notably, the ferritin vectors were capable of generating pools with higher specific productivities than the control.

F. Transfectant Isolates

Isolates were obtained by “picking” colonies from transfection experiment #2. “Picking” was accomplished by aspirating directly over a colony with a P200 Pipetman set at 50 μl. The aspirated colony was transferred first to a 48-well plate and then to a 6 well plate when there were a sufficient number of cells. Specific productivities were assessed in 6-well plates at near confluent to confluent cell densities using the 24-hour assay described above. 40-50 isolates were analyzed for each construct. The results are shown in FIG. 15, in which the isolates are presented in the order of their specific productivity for each SEAP expression construct. The scale of specific productivity is consistent between the panels for comparison.

The majority of the isolates (63%) from the pSEAP2 transfections did not express product above the limit of detection. The highest productivity from pSEAP2 in this experiment was 46 units per cell per day (relative value for comparison). In contrast only 28% of the isolates from the pFerX8SEAP transfections expressed product below the limit of detection and 44% had productivities above the highest pSEAP2 transfectant. The highest productivity from pFerX8SEAP in this experiment was 259 units per cell per day, more than five-fold higher than the highest productivity from pSEAP2. Although the pFerX9SEAP construct performed better than pSEAP2, it did not perform as well as pFerX8SEAP.

### EXAMPLE 3

Reduction of Vector Size.

In order to reduce the size of the vector for ease of use, 5′ and/or 3′ regions of the vector were deleted (TABLE 5). These deletions were tested as before using SEAP as a reporter. Approximately 30 isolates were tested from each of the plasmids shown in TABLE 5 as well as from the controls, pSEAP2 and pUC18 (10 isolates).

TABLE 5

| | Region | 5′ end of the | 3′ end of the | Size of the |
|---|---|---|---|---|
| Plasmid | deleted | deletion* | deletion* | plasmid (bp)** |
| pFerX8SEAP | none | | | 19340 |
| pFerX10SEAP | 5′ | 2513 | 7414 | 14439 |
| pFerX11SEAP | 3′ | 13727 | 17636 | 15431 |
| pFerX12SEAP | 5′ | 2513 | 7414 | 8042 |
| | 3′ | 12704 | 19101 | |

*The deletion end points are based on the pFerX8 sequence numbering
**The SEAP gene constitutes 1557 bp of the plasmid

The pFerX11SEAP vector performed similarly to the pFerX8SEAP vector, indicating that the ˜3.9 kb deletion in the 3′ region described in TABLE 5 was not detrimental. The pFerX10SEAP and pFerX12SEAP vectors did not perform as well as pFerX8SEAP, indicating that the ˜4.9 kb 5′ deletion described in TABLE 5 was detrimental to function.

### Equivalents

While this invention has been particularly shown and described with references to certain specific embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

1-21. (canceled)

22. A genetic vector for stable transfection and expression of a desired protein within eukaryotic cells comprising:
(a) distal 5′ flanking sequences of a eukaryotic locus;
(b) proximal 5′ regulatory sequences of a eukaryotic locus;
(c) at least a first insertion site for a first heterologous coding sequence; and
(d) proximal 3′ regulatory sequences effective for transcription termination of a eukaryotic locus;
wherein said sequences are operably joined in order (a)-(d) in a 5′ to 3′ orientation, with optional linker sequences between adjacent sequences; and wherein
(1) said distal 5′ flanking sequences comprise a sequence of at least 100 bases having at least 70% identity to a nucleotide sequence found between 20 bp and 100,000 bp 5′ of a transcriptional initiation site of a ferritin heavy chain locus; or
(2) said proximal 5′ regulatory sequences comprise a sequence of at least 20 bases having at least 70% identity to a nucleotide sequence found between 1 bp and 10,000 bp 5′ of a translational initiation codon of a ferritin heavy chain locus.

23. A genetic vector for stable transfection and expression of a desired protein within eukaryotic cells comprising:
(a) distal 5′ flanking sequences of a eukaryotic locus;
(b) proximal 5′ regulatory sequences of a eukaryotic locus;
(c) at least a first heterologous coding sequence encoding said desired protein; and
(d) proximal 3′ regulatory sequences effective for transcription termination of a eukaryotic locus;
wherein said sequences are operably joined in order (a)-(d) in a 5′ to 3′ orientation, with optional linker sequences between adjacent sequences; and wherein
(1) said distal 5′ flanking sequences comprise a sequence of at least 100 bases having at least 70% identity to a nucleotide sequence found between 20 bp and 100,000 bp 5′ of a transcriptional initiation site of a ferritin heavy chain locus; or
(2) said proximal 5′ regulatory sequences comprise a sequence of at least 20 bases having at least 70% identity to a nucleotide sequence found between 1 bp and 10,000 bp 5′ of a translational initiation codon of a ferritin heavy chain locus.

24. A genetic vector of claim 22 wherein said distal 5′ flanking sequences are derived from a ferritin heavy chain locus.

25. A genetic vector of claim 22 wherein said proximal 5′ regulatory sequences are derived from a ferritin heavy chain locus.

26. A genetic vector of claim 22 wherein said proximal 5′ regulatory sequences and said 5′ distal flanking sequences are derived from a ferritin heavy chain locus.

27. A genetic vector of claim 22 wherein said proximal 3′ regulatory sequences are derived from a ferritin heavy chain locus.

28. A genetic vector of claim 22 further comprising distal 3′ flanking sequences of a ferritin heavy chain locus.

29. A genetic vector of claim 22 wherein said insertion site for a heterologous sequence includes at least one restriction endonuclease site.

30. A genetic vector as in claim 29 wherein said insertion site for a heterologous sequence is a polylinker site including at least two restriction endonuclease sites.

31. A genetic vector of claim 22 wherein said proximal 5′ regulatory sequences include a eukaryotic intron sequence.

32. A genetic vector as in claim 31 wherein said eukaryotic intron sequence is derived from intron 1 of a ferritin heavy chain gene.

33. A genetic vector of claim 22 wherein said proximal 5′ regulatory sequences include untranslated exon sequences.

34. A genetic vector of claim 22 wherein said distal 5′ flanking sequences and said proximal 5′ regulatory sequences have a total length of between 1,000 and 10,000 bases.

35. A genetic vector of claim 22 wherein said proximal 3′ regulatory sequences and any distal 3′ flanking sequences have a total length of between 1,000 and 10,000 bases.

36. A genetic vector of claim 22 wherein said distal 5′ flanking sequences comprise a sequence having at least 80% identity to said nucleotide sequence found between 20 bp and 100,000 bp 5′ of a transcriptional initiation site of a ferritin heavy chain locus.

37. A genetic vector of claim 22 wherein said distal 5′ flanking sequences comprise a sequence having at least 90% identity to said nucleotide sequence found between 20 bp and 100,000 bp 5′ of a transcriptional initiation site of a ferritin heavy chain locus.

38. A genetic vector of claim 22 wherein said distal 5′ flanking sequences comprise a sequence having 100% identity to said nucleotide sequence found between 20 bp and 100,000 bp 5′ of a transcriptional initiation site of a ferritin heavy chain locus.

39. A genetic vector of claim 22 wherein said distal 5′ flanking sequences comprise a sequence of at least 500 bases having at least 70% identity to said nucleotide sequence found between 20 bp and 100,000 bp 5′ of a transcriptional initiation site of a ferritin heavy chain locus.

40. A genetic vector of claim 22 wherein said distal 5′ flanking sequences comprise a sequence of at least 1,000 bases having at least 70% identity to said nucleotide sequence found between 20 bp and 100,000 bp 5′ of a transcriptional initiation site of a ferritin heavy chain locus.

41. A genetic vector of claim 22 wherein said proximal 5′ regulatory sequences comprise a sequence having at least 80% identity to said nucleotide sequence found between 1 bp and 10,000 bp 5′ of a translational initiation codon of a ferritin heavy chain locus.

42. A genetic vector of claim 22 wherein said proximal 5′ regulatory sequences comprise a sequence having at least 90% identity to said nucleotide sequence found between 1 bp and 10,000 bp 5′ of a translational initiation codon of a ferritin heavy chain locus.

43. A genetic vector of claim 22 wherein said proximal 5′ regulatory sequences comprise a sequence having 100% identity to said nucleotide sequence found between 1 bp and 10,000 bp 5′ of a translational initiation codon of a ferritin heavy chain locus.

44. A genetic vector of claim 22 wherein said proximal 5′ regulatory sequences comprise a sequence of at least 500 bases having at least 70% identity to said nucleotide sequence found between 1 bp and 10,000 bp 5′ of a translational initiation codon of a ferritin heavy chain locus.

45. A genetic vector of claim 22 wherein said proximal 5′ regulatory sequences comprise a sequence of at least 1,000 bases having at least 70% identity to said nucleotide sequence found between 1 bp and 10,000 bp 5′ of a translational initiation codon of a ferritin heavy chain locus.

46. A genetic vector of claim 22 wherein the length of said first insertion site is 0, 1, 2 or 3 bp.

47. A genetic vector of claim 22 wherein the length of said first insertion site is 4 bp.

48. A genetic vector of claim 22 wherein the length of said first insertion site is 1,000 bp.

49. A genetic vector of claim 22 wherein the length of said first insertion site is 5,000 bp.

50. A genetic vector of claim 22 wherein said proximal 3′ regulatory sequences comprise a sequence having at least 70% identity to a nucleotide sequence found within the proximal 3′ regulatory sequences of a ferritin heavy chain locus.

51. A genetic vector of claim 22 wherein said proximal 3′ regulatory sequences comprise a sequence having at least 80% identity to a nucleotide sequence found within the proximal 3′ regulatory sequences of a ferritin heavy chain locus.

52. A genetic vector of claim 22 wherein said proximal 3′ regulatory sequences comprise a sequence having at least 90% identity to a nucleotide sequence found within the proximal 3′ regulatory sequences of a ferritin heavy chain locus.

53. A genetic vector of claim 22 wherein said proximal 3′ regulatory sequences comprise a sequence having 100% identity to a nucleotide sequence found within the proximal 3′ regulatory sequences of a ferritin heavy chain locus.

54. A genetic vector of claim 22 wherein said proximal 3′ regulatory sequences comprise at least 10 nucleotides.

55. A genetic vector of claim 22 wherein said proximal 3′ regulatory sequences comprise at least 1,000 nucleotides.

56. A genetic vector of claim 22 wherein said proximal 3′ regulatory sequences consist essentially of a polyadenylation signal.

57. A genetic vector of claim 22 further comprising a distal 3′ flanking sequence of a eukaryotic locus comprising a sequence of at least 100 bases having at least 70% identity to a nucleotide sequence found within the distal 3′ flanking sequences of a ferritin heavy chain locus.

58. A genetic vector of claim 57 wherein said distal 3′ flanking sequence comprises a sequence having at least 80% identity to said nucleotide sequence found within the distal 3′ flanking sequences of a ferritin heavy chain locus.

59. A genetic vector as in claim 57 wherein said distal 3′ flanking sequence comprises a sequence having at least 90% identity to said nucleotide sequence found within the distal 3′ flanking sequences of a ferritin heavy chain locus.

60. A genetic vector as in claim 57 wherein said distal 3′ flanking sequence comprises a sequence having 100% identity to said nucleotide sequence found within the distal 3′ flanking sequences of a ferritin heavy chain locus.

61. A genetic vector as in claim 57 wherein said distal 3′ flanking sequence comprises a sequence of at least 500 bases having at least 70% identity to said nucleotide sequence found within the distal 3′ flanking sequences of a ferritin heavy chain locus.

62. A genetic vector as in claim 57 wherein said distal 3′ flanking sequence comprises a sequence of at least 1,000 bases having at least 70% identity to said nucleotide sequence found within the distal 3′ flanking sequences of a ferritin heavy chain locus.

63. The genetic vector pFerX8.

64. The genetic vector pFerX11.

65. A eukaryotic cell transfected with a vector of claim 22.

66. A eukaryotic cell of claim 65 wherein said vector has stably integrated into a chromosome of a said cell.

67. A eukaryotic cell of claim 65 wherein said first coding sequence is expressed in said cell.

68. A eukaryotic cell comprising:
(a) distal 5′ flanking sequences of a eukaryotic locus;
(b) proximal 5′ regulatory sequences of a eukaryotic locus;
(c) at least a first coding sequence; and
(d) proximal 3′ regulatory sequences effective for transcription termination of a eukaryotic locus;
wherein said sequences are operably joined in order (a)-(d) in a 5′ to 3′ orientation, with optional linker sequences between adjacent sequences; and wherein (1) said distal 5′ flanking sequences comprise an exogenous sequence of at least 100 bases having at least 70% identity to a nucleotide sequence found between 20 bp and 100,000 bp 5′ of a transcriptional initiation site of a ferritin heavy chain locus; or
(2) said proximal 5′ regulatory sequences comprise an exogenous sequence of at least 20 bases having at least 70% identity to a nucleotide sequence found between 1 bp and 10,000 bp 5′ of a translational initiation codon of a ferritin heavy chain locus.

69. A eukaryotic cell comprising:
an exogenous 5′ distal flanking sequence derived from a ferritin heavy chain locus operably joined to a coding sequence.

70. A method of producing a desired protein in a eukaryotic cell comprising:
(a) providing at least one cell of claim 65 or a descendent thereof;
(b) maintaining said cell in a culture under conditions which permit high expression of said desired protein; and
(c) isolating said desired protein from said culture.
